(19) United States
(12) Patent Application Publication
(10) Pub. No.: US 2002/0006632 A1
(43) Pub. Date: Jan. 17, 2002

(54) Biosensor

(76) Inventors: Gopalakrishnakone Ponnampalam, Singapore (SG); Emmanuel Selvanayagam Zachariah, Singapore (SG); Sridhar Uppili, Singapore (SG); Neuzil Pavel, Singapore (SG)

Correspondence Address: BIRCH STEWART KOLASCH & BIRCH, PO BOX 747, FALLS CHURCH, VA, 22040-0747, US

(21) Appl. No.: 09/790,520

(22) Filed: Feb. 23, 2001

(30) Foreign Application Priority Data
Feb. 24, 2000 (SG) 20001027-2

(51) Int. Cl. A61K 39/395; G01N 33/53; G01N 33/537; G01N 33/543

(52) U.S. Cl. 435/7.92; 438/1; 424/146.1

(57) ABSTRACT

An improved biosensor is disclosed comprising an immobilised membrane adhering to a pH sensitive surface of an ion-sensitive field effect transistor by a polysiloxane matrix and comprising an analyte detection agent for detecting and/or quantifying a target analyte. The improvement resides in the immobilised membrane having a thickness of less than about 100 nm which, when compared to conventional immunochemical membranes having a thickness of between 500 nm and 2.0 μm, has reduced propensity for antibody aggregation with improved antibody affinity and sensitivity of the sensor.

### FIELD OF INVENTION

The present invention relates generally to biosensors. More particularly, the invention relates to an improved ion-sensitive field effect transistor (ISFET) based biosensor having an immobilised thin film membrane of less than about 100 nm comprising an agent therein for detection and/or quantification of a target analyte of clinical and/or medicolegal importance.

### BACKGROUND OF THE INVENTION

Diagnostic tools for detecting and/or quantifying biological analytes are generally based on specific binding interactions between a ligand and a receptor (i.e., a specific binding pair). Specific binding pairs used commonly in diagnostics include antigen-antibody, hormone-receptor, drug-receptor, cell surface antigen-lectin, biotin-avidin, and complementary nucleic acid strands. The analyte for detection may be either member of the specific binding pair; alternatively, the analyte may be a ligand analog that competes with the ligand for binding to the complementary receptor.

Numerous methods for detecting and/or quantifying ligand/receptor interactions have been developed. The simplest of these is a solid-phase format (e.g., radioimmunoassay) employing a reporter-labelled ligand whose binding to or release from a solid surface is triggered by the presence of analyte ligand or receptor. In a typical solid-phase sandwich type assay (e.g., enzyme-linked immunosorbent assay or ELISA), the analyte to be measured is a ligand with two or more binding sites, allowing ligand binding both to a receptor, e.g., antibody, carried on a solid surface, and to a reporter-labelled second receptor. The presence of analyte is detected (or quantified) by the presence (or amount) of reporter bound to the solid surface. In a typical solid-phase competitive binding assay, an analyte ligand (or receptor) competes with a reporter-labelled analyte analog for binding to a receptor (or ligand) carried on a solid support. The amount of reporter signal associated with the solid support is inversely proportional to the amount of sample analyte to be detected or determined. Unfortunately, such conventional methods suffer from numerous disadvantages in that they require expensive equipment and sophisticated techniques that must be carried out by highly trained personnel.

Recently, a variety of electrochemical biosensors have been developed for facile, point-of-care detection and/or quantification of ligand receptor binding events. Generally, a biosensor is composed of (i) a biochemical receptor, which uses receptors such as enzymes, antibodies or microbes to detect an analyte, and (ii) a transducer, which transforms changes in physical or chemical value accompanying the reaction into a measurable response, most often an electrical signal [3]. Several biosensors based on immobilised enzymes are available commercially and are especially useful in clinical analysis [4]. The term immunosensor is used when an antibody or antigen is immobilised to interact respectively with its specific binding partner (i.e., a target antigen or a target antibody) [5].

The conversion of the biological recognition (binding) event to a quantitative result has been accomplished by a variety of techniques, including electrochemical, calorimetric and optical detection [6]. Of the electrochemical technologies for biosensors, the ion-sensitive field effect transistor (ISFET) has been the centre of special attention as transducer. ISFETs were introduced in 1970, and were the first type of this class of sensor in which a chemically sensitive layer was integrated with solid state electronics [7]. By excluding the gate metal in a FET and using a pH sensitive gate insulator, a pH sensitive ISFET could be constructed [8].

After development of the ISFET many different types of field effect transistor (FET) based sensors have been presented. The application of enzymes as the selecting agent in ISFET based sensing systems has lead to the development of highly selective sensors. Such enzyme-modified ISFETs (EnFETs) can in principle be constructed with any enzyme that produces a change in pH on conversion of the relevant enzyme substrate [9]. In one example of an EnFET disclosed by Johnson et al (U.S. Pat. No. 4,020,830), the gate region of a FET is overlaid with a membrane (the pH sensitive gate insulator) capable of interacting selectively with ions present in a solution. That is, the membrane adsorbs ions from the solution which ions alter the electric potential of the membrane and therefore of the gate. A second thin film layer or membrane, having an enzyme or substrate immobilised therein is positioned over the ion-selective membrane. When the membrane containing the enzyme, for example, is contacted with a solution containing the substrate, the substrate diffuses into this membrane and reacts with the enzyme. A net yield or loss of ions accompanies the reaction. The ion concentration of the underlying ion-selective membrane then changes, thereby affecting its electric potential and giving rise to a measurable change in an electrical signal.

EnFETs have been adapted for use as immunosensors. In such “immunoFETs”, an antibody or antigen is immobilised in the second thin film membrane described above in place of the enzyme or substrate [10, 11]. In the competitive binding assay format, a sample antigen competes with enzyme labelled antigen for the antibody-binding sites on the membrane. The membrane is then washed, and the immunoFET is placed in a solution containing a substrate for the enzyme [12]. Enzyme immunoFETs based on the sandwich assay have also been described [13] in which an antibody is used to capture an analyte-antigen and an enzyme-labelled second antibody is used to detect/quantify the captured analyte-antigen. After removal of the non-specifically adsorbed second antibody, the immunoFET is placed into a substrate-containing solution and the extent of the enzymatic reaction is monitored electrochemically.

ImmunoFETs have several advantages over conventional solid phase enzyme immunoassays. These include the considerable miniaturisation of the device, the low cost of the transducer and the rapidity of sensor response. Prototype immunoFET devices (see Aizawa et al, 1977, J. Membr. Sc. 2: 125; and Janata and Huber, 1980, In “Ion-sensitive Electrodes in Analytical Chemistry” (Freiser H. ed.), 2: 107-174, Plenum Press), unfortunately, were deficient in that their immunochemical membranes were relatively unstable and prone to interference by other chemical species such as ions or proteins present in solution.

More recent immunoFET devices have been described with improved stability and reduced chemical interference. For example, reference may be made to Collapicchioni et al (U.S. Pat. No. 5,160,597) who describe an immunoFET device in which stability of the immunochemical membrane is improved by bonding of the membrane to the surface silicon oxide (pH sensitive gate insulator) of the device by a polysiloxane matrix. Formation of this matrix is achieved by deposition of a siloxane prepolymer on the surface silicon oxide using spin-on techniques or plasma deposition followed by thermal curing of the prepolymer. Collapicchioni et al emphasise that the matrix must have a thickness of between 0.5 and 3 μm whereas the thickness of the immunochemical membrane can be between 0.5 and 2 μm.

Despite improvements in immunoFET design, one disadvantage still persists in that antibodies have a tendency to aggregate in the immunochemical membrane of the immunoFET, which reduces the affinity of antibody for its antigen, and which in turn reduces the sensitivity of the sensor.

In work leading up to the present invention, the inventors sought to provide improved methods and devices for detection of snake envenomation, which remains an important heath and medico-legal problem in many parts of the world, especially in developing countries [1]. Identification of the biting species of a snake by the victims is usually difficult and clinical manifestations alone are not reliable because of overlapping symptoms. Development of a simple, reliable, rapid, inexpensive as well as field executable detection kit for venoms and toxins is of enormous significance. Many immunoassays such as immunodiffusion, immunoelectrophoresis, immunofluorescence, haemagglutination and radioimmunoassay have been developed in the last two decades. However, high cost and low sensitivity have made these methods unattractive for the detection of venoms. ELISA appears to be the preferred method at the present time for detection of venoms and toxins. However some versions of ELISA in current use still lack the required specificity, are too slow for treatment and/or are too expensive for routine use [2].

### SUMMARY OF THE INVENTION

While investigating immunoFETs as platforms for detection of snake envenomation, the present inventors discovered unexpectedly that by decreasing the thickness of the immunochemical membrane of an immunoFET device from a conventional range of between 0.5 μm and 2.0 μm to less than about 100 nm, a marked reduction in antibody aggregation results with improved antibody affinity and sensitivity of the sensor.

Accordingly, in one aspect of the invention, there is provided a biosensor comprising an immobilised membrane adhering to a pH sensitive surface of an ion-sensitive field effect transistor by a polysiloxane matrix and comprising an analyte detection agent for detecting and/or quantifying a target analyte, the polysiloxane matrix being chosen from functional organosilanes of general formula:

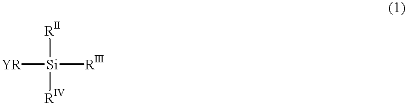

where R^{II}, R^{III}, and R^{IV}, which can be equal or different, are C₁-C₁₀ alkyl or alkoxy groups, R=(CH₂)ₘX(CH₂)ₙ

where X is CH₂ or a mono or polycondensed aromatic group or NH or O, m and n, which can be equal or different, are whole numbers between 0 and 10, but not 0 when X is NH or O,

Y can be —NH₂ or —OH or —SH, or from functional organosilanes of general formula:

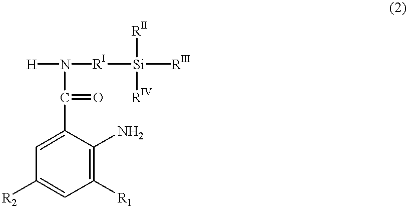

in which R₁ and R₂, which can be equal or different, are Cl, Br, CH₃, NO₂, NH₂ or H, R^{II}, R^{III}, and R^{IV}, which can be equal or different, are C₁-C₁₀ alkyl or alkoxy groups, R^{I} can be a C₁-C₁₀ alkyl, aminoalkyl, aminoalkylaryl or alkylaryl group,

characterised in that the thickness of said membrane is less than about 100 nm.

Preferably, the immobilised membrane has a thickness of between about 10 nm and about 100 nm, more preferably of between about 30 nm to about 90 nm, and still more preferably of between about 50 nm to about 80 nm.

Suitably, the polysiloxane matrix has a thickness of between about 10 nm and about 80 nm, preferably of between about 20 nm to about 70 nm, and more preferably of between about 30 nm to about 60 nm.

Preferably, the pH sensitive surface is formed of a member selected from the group consisting of aluminium oxide, silicon oxide, silicon nitride or tantalum pentoxide.

Preferably, but not exclusively, the analyte detection agent is selected from the group consisting of an antigen and an antigen-binding molecule.

In a preferred embodiment, the analyte detection agent is an antigen-binding molecule. In such a case, the target analyte is preferably an antigen selected from the group consisting of a venom and a toxin.

Preferably, the toxin is a bungarotoxin, preferably a β-bungarotoxin (β-BuTx).

In another aspect, the invention resides in a process of forming a said device as broadly described above, said process comprising:

applying a siloxane prepolymer to the pH sensitive surface of an ISFET-type device;

blowing excess siloxane prepolymer from said surface;

curing the siloxane prepolymer such that polymerisation of the silane alkoxy groups of the prepolymer takes place by hydrolysis to obtain a polysiloxane matrix;

adhering the matrix to the pH sensitive surface by reaction of other alkoxy groups with hydroxyl groups present on said surface; and

reacting an analyte detection agent with the aliphatic amino groups present on the polysiloxane matrix.

Alternatively, the process may comprise:

applying a siloxane prepolymer to the pH sensitive surface of an ISFET-type device;

blowing excess siloxane prepolymer from said surface;

curing the siloxane prepolymer such that polymerisation of the silane alkoxy groups of the prepolymer takes place by hydrolysis to obtain a polysiloxane matrix;

adhering the matrix to the pH sensitive surface by reaction of other alkoxy groups with hydroxyl groups present on said surface;

activating the aliphatic amino groups present on the polysiloxane matrix by bifunctional coupling agents; and

reacting an analyte detection agent with the activated amino groups of the polysiloxane matrix.

Preferably, the step of blowing is characterised in that a jet of compressed gas is used to blow the excess siloxane prepolymer from the pH sensitive surface.

Suitable gases include, but are not restricted to nitrogen, a noble gas such as helium or argon, and air.

Preferably, the jet of compressed gas is blown at an angle of between 10 degrees and 70 degrees, and more preferably of between 30 degrees and 50 degrees, to the said surface.

In a further aspect according to the invention, there is provided an antigen-binding molecule that is immuno-interactive with a bungarotoxin, preferably a β-bungarotoxin.

In yet another aspect, there is provided a method of detecting the presence or absence of a bungarotoxin, and more preferably a β-bungarotoxin, in a patient, comprising:

isolating a biological sample from the patient, contacting the biological sample with an antigen-binding molecule that is immuno-interactive with said bungarotoxin, and detecting the presence of a complex comprising the said antigen-binding molecule and the bungarotoxin.

Preferably, the antigen-binding molecule is an anti-bungarotoxin monoclonal antibody.

The invention also extends to the use of the improved biosensor as broadly described above in a kit for detecting and/or measuring a target antigen in a biological sample, and to the use of the antigen-binding molecule as broadly described above in a kit for detecting and/or quantifying a bungarotoxin in a biological sample.

In another aspect, the invention provides a composition for use in treatment or prophylaxis of envenomation, comprising an antigen-binding molecule as broadly described above, together with a pharmaceutically acceptable carrier.

Suitably, the envenomation is caused by a Bungarus species, preferably Bungarus multicinctus.

### BRIEF DESCRIPTION OF THE DRAWINGS

FIG. 1 is a graph showing reactivity of different mAbs with native β-BuTx.

FIG. 2 is an HPLC elution profile of A and B chains of β-BuTx.

FIG. 3 is a graphical representation of the reactivity of mAbs 5 (Panel A), 11 (Panel B) and 15 (Panel C) with native β-BuTx, A and B chains of the toxin.

FIG. 4 is a sensogram of competition binding analysis using different mAbs.

FIG. 5 is an SDS-PAGE profile of mAb 15. Lane 1, ammonium sulphate precipitated (reduced) mAb 15; Lane 2, affinity purified (reduced) mAb 15; Lane 3, affinity purified (non-reduced) mAb 15; Lane 4, molecular weight markers.

FIG. 6 is a graph showing the specificity of mAb 15.

FIG. 7 is a graph showing a standard curve for quantitation of β-BuTx.

FIG. 8 is a schematic showing a cross section of an ISFET.

FIG. 9 is a graph showing the time response of an ISFET for quantitation of 0.5 μg/mL of β-BuTx.

FIG. 10 is a graph showing ISFET response to pH change.

FIG. 11 is a graph showing I_{D} vs. V_{REF} characteristic of an ISFET.

FIG. 12 is a graph showing I_{D} vs. V_{DS} characteristic of an ISFET.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to a thickness of a layer or membrane that varies by as much as 30%, preferably by as much as 20%, and more preferably by as much as 10% to a reference thickness.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

The term “biological sample” as used herein refers to a sample that may be extracted, untreated, treated, diluted or concentrated from a patient. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. The biological sample preferably includes serum, whole blood, plasma, lymph and ovarian follicular fluid as well as other circulatory fluid and saliva, mucus secretion and respiratory fluid. More preferably, the biological sample is a circulatory fluid such as serum or whole blood or a fractionated portion thereof. Most preferably, the biological sample is serum or a fractionated portion thereof.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes).

As used herein, a “specific binding pair” comprises two different molecules wherein one of the molecules through chemical or physical means specifically binds to a second molecule. Such specific binding partners, examples of which are described in U.S. Pat. No. 5,075,078, include antigens and antibodies, lectins and carbohydrates, complementary peptides, protein, carbohydrate and nucleic acid structures, enzyme inhibitors and enzymes, Protein A and IgG as well as effector and receptor molecules.

As used herein “target analyte” refers to any substance that is required to be detected or quantified, including an antigenic substance, a hapten, an antibody, a protein, a peptide, an amino acid, a nucleic acid, a hormone, a steroid, a vitamin, a carbohydrate, a lipid, a blood clotting factor, a pathogenic organism for which polyclonal and/or monoclonal antibodies can be produced, a natural or synthetic chemical substance, a contaminant, a drug including those administered for therapeutic purposes as well as those administered for illicit purposes, and metabolites of or antibodies to any of the above substances. Preferably, the target analyte has two binding sites each of which is capable of forming a specific binding pair with a specific binding partner. However, it will be understood that it is sufficient for the target analyte to have a single binding site for a specific binding partner if the target analyte, in combination with a first specific binding partner, is capable of producing a unique binding site for a second specific binding partner. The target analyte is suitably a specific binding partner of the analyte detection agent.

2. ImmunoFET of the invention

The present invention provides an improved biosensor comprising an immobilised immunochemical membrane which adheres to a pH sensitive surface of an ion-sensitive field effect transistor by a polysiloxane matrix being chosen from functional organosilanes. The improvement resides in the immobilised membrane having a thickness of less than about 100 nm. The inventors have found that, when compared to conventional immunochemical membranes having a thickness of between 500 nm and 2.0 μm, the immunochemical membrane of the invention has reduced propensity for antibody aggregation with improved antibody affinity and sensitivity of the sensor.

Preferably, the immobilised membrane has a thickness of between about 10 nm and about 100 nm, more preferably of between about 30 nm to about 90 nm, and still more preferably of between about 50 nm to about 80 nm.

The inventors have also found that, compared to conventional matrices of the prior art, a substantially thinner matrix is advantageous for facilitating a thin immobilised immunochemical membrane, and for faster ion travel which provides a more rapid response to change in pH. Accordingly, it is preferable that the polysiloxane matrix has a thickness of less than 100 nm but is preferably in a range of between about 10 nm and about 80 nm, more preferably of between about 20 nm to about 70 nm, and still more preferably of between about 30 nm to about 60 nm.

Silanes for preparing the polysiloxane matrix of the present invention are preferably selected from the group consisting of:

3-aminopropyltriethoxysilane, H₂N(CH₂)₃Si(OC₂H₅)₃

aminomethyltriethoxysilane, H₂NCH₂Si(OC₂H₅)₃

2-aminoethyl-aminopropyltriethoxysilane, H₂N(CH₂)₂NH(CH₂)₃Si(OCH₃)₃

2-aminoethyl-aminopropyltriethoxysilane, H₂N(CH₂)₂NH(CH₂)₃Si(OC₂H₅)₃

2-aminoethyl-aminopropylmethyldimethoxysilane.

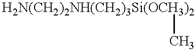

The immunochemical membrane comprises an analyte detection agent, which preferably comprises an antigen or an antigen-binding molecule. Preferably, the analyte detection agent is an antigen-binding molecule.

Any antigen-binding molecule is contemplated that is immuno-interactive with the target analyte. For example, the antigen-binding molecule may comprise whole polyclonal antibodies. Such antibodies may be prepared, for example, by injecting an antigen into a production species, which may include mice or rabbits, to obtain polyclonal antisera. Methods of producing polyclonal antibodies are well known to those skilled in the art. Exemplary protocols which may be used are described for example in Coligan et al, CURRENT PROTOCOLS IN IMMUNOLOGY, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975, Nature 256, 495-497), or by more recent modifications thereof as described, for example, in Coligan et al., (199 1, supra) by immortalising spleen or other antibody producing cells derived from a production species which has been inoculated with an antigen.

The invention also contemplates as antigen-binding molecules Fv, Fab, Fab′ and F(ab′)₂ immunoglobulin fragments.

Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (Krebber et al. 1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plückthun et al (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond therebetween. Suitable methods of producing dsFv are described for example in (Glockscuther et al. Biochem. 29: 1363-1367; Reiter et al. 1994, J. Biol. Chem. 269: 18327-18331; Reiter et al. 1994, Biochem. 33: 5451-5459; Reiter et al. 1994. Cancer Res. 54: 2714-2718; Webber et al. 1995, Mol Immunol. 32: 249-258).

Also contemplated as antigen-binding molecules are single variable region domains (termed dAbs) as for example disclosed in (Ward et al. 1989, Nature 341: 544-546; Hamers-Casterman et al. 1993, Nature. 363: 446-448; Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (i.e., having more than one antigen binding site). Such multivalent molecules may be specific for one or more antigens. Multivalent molecules of this type may be prepared by dimerisation of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerisation may be facilitated by fusion of the antibody fragments to amphiphilic helices that naturally dimerise (Pack P. Plunckthun, 1992, Biochem. 31: 1579-1584), or by use of domains (such as the leucine zippers jun and fos) that preferentially heterodimerise (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

In a preferred embodiment, the antigen-binding molecule is immuno-interactive with an antigen selected from the group consisting of a venom and a toxin. The venom or toxin may be obtained from any suitable venom- or toxin-producing animal including, but not restricted to, snakes, insects and fish. In a preferred embodiment, the venom or toxin is obtained from a snake, and preferably from an Elaphidae species. Preferably, the venom or toxin is obtained from a Bungarus species, and more preferably from Bungarus multicinctus. Suitably, the toxin is a neurotoxin, preferably a bungarotoxin, and more preferably a β-bungarotoxin.

In a preferred embodiment, the antigen binding molecule is a monoclonal antibody that is immuno-interactive with a bungarotoxin, preferably a β-bungarotoxin. Thus, the invention, in another aspect, also provides an antigen-binding molecule that is immuno-interactive with a bungarotoxin, preferably a β-bungarotoxin.

For a typical “two-site or sandwich assay”, a first analyte detection agent is preferably immobilised in the immunochemical membrane of the invention, which binds to a target analyte that may be present in a biological sample to form a complex. A second analyte detection agent is also provided, which binds to a second site of the target analyte, or to the first analyte detection agent. The second analyte detection agent is preferably associated with an enzyme that catalyses a reaction in which ions are formed from neutral molecules. The enzyme is suitably conjugated with the analyte detection agent. Suitable enzymes which may be used in this connection include, but are not restricted to, urease, penicillinase, esterases, hydrolases, amino acid oxidase and glucose oxidase. In a preferred embodiment, the enzyme is urease and the substrate is urea.

In another aspect, the invention resides in a process of forming the improved biosensor as broadly described in Section 2, comprising:

applying a siloxane prepolymer on the pH sensitive surface of an ISFET-type device;

blowing excess siloxane prepolymer from said surface;

curing the siloxane prepolymer such that polymerisation of the silane alkoxy groups of the prepolymer takes place by hydrolysis to obtain a polysiloxane matrix;

adhering the matrix to the pH sensitive surface by reaction of other alkoxy groups with hydroxyl groups present on the surface; and

reacting an analyte detection agent with the aliphatic amino groups present on the polysiloxane matrix.

Alternatively, the process may comprise:

applying a siloxane prepolymer on the pH sensitive surface of an ISFET-type device;

blowing excess siloxane prepolymer from said surface;

curing the siloxane prepolyrner such that polymerisation of the silane alkoxy groups of the prepolymer takes place by hydrolysis to obtain a polysiloxane matrix;

adhering the matrix to the pH sensitive surface by reaction of other alkoxy groups with hydroxyl groups present on the surface;

activating the aliphatic amino groups present on the polysiloxane matrix by a bifunctional coupling agent; and

reacting an analyte detection agent with the activated amino groups of the polysiloxane matrix.

The silane prepolymer may be applied to the pH sensitive surface of an ISFET-type device by any suitable technique including deposition of the prepolymer by solution casting, spin-on techniques or plasma deposition as is known in the art. Preferably, the silane prepolymer is applied by immersion of said pH sensitive surface (e.g., by dip-coating) in a solution of said prepolymer.

Preferably, a jet of compressed gas is used to blow the excess siloxane prepolymer from the pH sensitive surface. Suitably, the compressed gas includes, but is not restricted to nitrogen, a noble gas such as helium or argon, and air. The jet of compressed gas is preferably blown at an angle of between 10 degrees and 70 degrees, and more preferably of between 30 degrees and 50 degrees, to the said surface.

The bifunctional coupling agent used in the second-mentioned process can be chosen from dialdehydes (preferably glutaraldehyde) or from diisocyanates (such as toluene 2.4-diisocyanate).

Curing of the siloxane prepolymer may be effected by any suitable technique but is preferably effected by thermal curing at a temperature of between 80° and 140° C.

Immobilisation of the analyte detection agent by reaction with the polysiloxane matrix may be carried out by any suitable process (e.g., adsorption, occlusion in matrix, crosslinking with bifunctional coupling agents or covalent bonding to a carrier). The analyte detection agent (e.g., an antigen) may require functionalisation in order to render it reactable with the matrix. Suitably, the analyte detection agent is immobilised with a bifunctional coupling agent and preferably with the bifunctional coupling agent used to activate the aliphatic amino groups present on the polysiloxane layer. Preferably, the analyte detection agent is reacted with the activated polysiloxane matrix by immersion of the matrix (e.g., dip coating) in a solution containing the analyte detection agent. A preferred solution containing the analyte detection agent is, for example, one which has been adjusted to pH 7.0 with a phosphate buffer. The operation of immersing the polysiloxane matrix in the analyte detection agent-containing solution must be carefully conducted so that the analyte detection agent is bound or fixed onto every functional group of the film in a highly dense and homogeneous state in order to prevent the adsorption of any non-specific substance, i.e., reactions other than the target analyte-analyte detection agent reaction which is to be detected by the immunosensor. The prevention of such non-specific adsorption may be enhanced by the treatment of the immunochemical membrane with a suitable agent such as BSA (bovine serum albumin), or monoethanolamine.

3. Detection of bungarotoxins

The presence or absence of a bungarotoxin, and more preferably a β-bungarotoxin, in a patient may be determined by isolating a biological sample from the patient, contacting the biological sample with an antigen-binding molecule as described in Section 2, and detecting the presence of a complex comprising the said antigen-binding molecule and the bungarotoxin. In this regard, the antigen-binding molecule may be species-specific, that is specific to a bungarotoxin of a particular Bungarus species. Preferably, the species is Bungarus multicinctus. Suitably, the antigen-binding molecule detects a bungarotoxin from a plurality of Bungarus species.

Any suitable technique for determining formation of the complex may be used. In a preferred embodiment, detection and/or quantification of the complex is determined by use of the improved biosensor of the invention.

Alternatively, an antigen-binding molecule according to the invention, having a reporter molecule associated therewith may be utilised in other non-FET based immunoassays including, but are not restricted to, radioimmunoassays (RIAs), enzyme-linked immunosorbent assays (ELISAs) and immunochromatographic techniques (ICTs), Western blotting which are well known those of skill in the art. For example, reference may be made to “CURRENT PROTOCOLS IN IMMUNOLOGY” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including serum, whole blood, and plasma or lymph fluid. The sample is, therefore, generally a circulatory sample comprising circulatory fluid.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al), 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.

4. Compositions

A further feature of the invention is the use of the antigen-binding molecules of the invention (“therapeutic agents”) as actives, together with a pharmaceutically acceptable carrier, in a composition for protecting or treating patients against envenomation by a Bungarus species, preferably Bungarus multicinctus.

Depending upon the particular route of administration, a variety of pharmaceutically acceptable carriers, well known in the art may be used. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Any suitable route of administration may be employed for providing a mammal or a patient with a composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of a therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Compositions suitable for oral or parenteral administration may be presented as discrete units such as capsules, sachets or tablets each containing a pre-determined amount of one or more immunogenic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the immunogenic agents of the invention with liquid carriers or finely divided solid carriers or both, and then, if necessary, shaping the product into the desired presentation.

The above compositions may be administered in a manner compatible with the dosage formulation, and in such amount as is therapeutically effective. The dose of therapeutic agent administered to a patient should be sufficient to effect a beneficial response in the patient over time such as a reduction in the level of bungarotoxin or to ameliorate the condition to be treated. The quantity of the therapeutic agent(s) to be administered may depend on the subject to be treated inclusive of the age, sex, weight and general health condition thereof. In this regard, precise amounts of the therapeutic agent(s) for administration will depend on the judgement of the practitioner. In determining the effective amount of the therapeutic agent to be administered in the treatment or prophylaxis of the condition associated with envenomation, the physician may evaluate circulating plasma levels, progression of the condition, and the production of anti-bungarotoxin antibodies.

In any event, those of skill in the art may readily determine suitable dosages of the immunogenic and therapeutic agents of the invention. Such dosages may be in the order of nanograms to milligrams of the therapeutic agents of the invention.

5. Detection kits

The present invention also provides kits for the detection and or quantification of a target analyte in a biological sample.

In one embodiment, the kit may comprise an improved biosensor as broadly described in Section 2, together with a second analyte detection agent, which preferably comprises an enzyme that catalyses a reaction in which ions are formed from neutral molecules.

In another embodiment, the kit may comprise an antigen-binding molecule as broadly described in Section 2.

The kits may also optionally include appropriate reagents for detection of reporter molecules, positive and negative controls, washing solutions, dilution buffers and the like.

In order that the invention may be readily understood and put into practical effect, particular preferred embodiments will now be described by way of the following non-limiting examples.

### EXAMPLE 1

Production and Characterisation of Monoclonal Antibodies

Methods

Immunization of mice with β-BuTx

In order to get donor splenocytes for the fusion, Balb/c mice were injected with native β-BuTx and the mouse, which showed high titre antibodies against the toxin, was selected for fusion. Eight Balb/c mice (18-20 g) were immunised by subcutaneous (s.c.) injection of 1 μg of native β-BuTx emulsified in 200 μL Freund's complete adjuvant. Booster s.c. injections of 3 μg toxin in PBS, pH 7.4 emulsified with Freund's incomplete adjuvant were given 30 and 60 days after priming. Seven days later, individual serum antibody responses were evaluated by ELISA and the best responding animal was selected as donor of spleen cells. Four days before fusion, the mouse received 3 μg of toxin in 200 μl of PBS, pH 7.4 by s.c. route with out adjuvant.

Production of hybridomas and monoclonal antibodies

Splenocytes harvested from the hyperimmunised mice were fused with P3.×63. Ag8. U1 (P3 U 1) myeloma cells (4:1) in the presence of 50% (w/v) PEG 4000 [16]. The cells (1.5×10⁵) were plated in IMDM medium supplemented with 10% (v/v) foetal calf serum and HAT on a feeder layer of Balb/c mouse thymocytes (2×10⁵ cells) in 96 well plates and were maintained therein until colonies had formed. HAT medium were then replaced by aminopterin-free medium (HT medium). Antitoxin occurrence was tested by ELISA screening procedure. The wells containing hybridomas secreting mAb specific to β-BuTx were subcloned by limiting dilution, at an average cell density 0.3-1 cell per well. The wells with a single colony were selected for subsequent development.

Preparation of ascitic fluid

For mass production of mAb, ascitic fluids were produced by injecting intraperitoneally (i.p.) 5×10⁶ hybridoma cells into Balb/c mice that had previously been given 0.5 mL pristane. One to two weeks later, ascitic fluid was withdrawn and the antibodies were separated by centrifugation.

Purification of monoclonal antibodies

MAbs from tissue culture medium/ascitic fluids were concentrated by precipitation in 40% saturated ammonium sulfate solution. The IgG fraction was purified by the affinity chromatography on Hi trap protein G column according to manufacturer's instructions (Pharmacia, Sweden).

Sodium dodecyl sulphate-polyacrylamide gel electrophoresis (SDS-PAGE)

Five micrograms of mAb 15 was subjected to electrophoresis on a 10% gel and the resolved proteins were visualised by staining with Coomassie brilliant blue R 250. The molecular weight of proteins was assessed by running standard marker proteins (200, 99, 66,45 and 29 kDa) in parallel lanes.

Determination of immunoglobulin class/subclass

The Ouchterlony double-diffusion technique was employed using rabbit anti-mouse IgG1, IgG2a, IgG2b, IgG3, IgGA and IgM (Cappel). The results were confirmed using a commercially available mouse mAb isotyping kit (IsoStrip™, Boehringer Mannheim, Germany).

Specificity of mAb 15

Cross-reactivity of mAb 15 towards other venoms was tested by ELISA. Crude venoms of phylogenitically related (Bungarus caeruleus, Naja naja) and unrelated (Echis carinatus, Vipera russelli) snakes were coated on to microtiter wells (5 μg/mL) and the wells were incubated with mAb 15 (1:100). HRP conjugated rabbit anti-mouse antibodies were used as the secondary antibodies and ortho-phenylenediamine (OPD) was used as the substrate.

ELISA

The wells of polyvinyl chloride plates (PVC) (Dynatech) were coated with 100 μL of native β-BuTx or A chain or B chain (5 μg/mL) diluted in carbonate-bicarbonate buffer, pH 9.6. After blocking with 1% skim-milk solution in PBS, undiluted supernatants from the hybrid-containing well/ascitic fluids/mAbs were added to each well. The plates were incubated with HRP-conjugated anti-mouse immunoglobulin (Dako-patts, Denmark) diluted to 1:2000 in 0.5% bovine serum albumin (BSA) in PBS-Tween. OPD was used as a substrate for the enzyme with 2.5 M H₂SO₄ as stopping solution. Optical density values were read with a microELISA™ reader (Dynatech) at 490 nm.

Separation and characterisation of A and B chains of β-BuTx

In order to identify the epitopes recognised by the three mAbs, the interchain disulfide bond between A and B chain of the toxin was reduced and the cleavage product was separated by HPLC as follows. Ten microgram of native β-BuTx was dissolved in 200 μl of 0.25 M Tris-HCl buffer (pH 8.5) containing 6 M guanidine-HCl and 1 mM EDTA, then 2 μl of 10% β-mercaptoethanol was added. After flushed with N₂, the reaction was allowed to proceed at 37° C. for 2 h under nitrogen. Two microliters of 4-vinylpyridine was mixed and incubated at room temperature for 2 h under nitrogen. The cleavage product was separated immediately by RP-HPLC on a Vydac C8 column (2.1×150 mm). The N-terminal amino acid sequences of A and B chains were determined by automatic Edman degradation using an Applied Biosystems 477A pulsed liquid-phase sequencer equipped with an on-line 120A PTH-amino acid analyser.

Epitope analysis by ELISA

ELISA was carried out to ascertain the location of epitopes recognised by the three mAbs 5, 11 and 15.

Epitope analysis by BIAcore™ system

The instrument (BIAcore™ system) and reagents for interaction analysis were obtained from Pharmacia Biosensor, Uppsala, Sweden. Immobilisation of β-BuTx to CM 5 sensor chip, via primary amine groups was performed according to manufacturer's instructions. Immobilisation of β-BuTx was performed with 30 μl of toxin (1 mg/ml) solubilised in 10 mM citrate buffer, pH 4.5 and injected at a flow rate of 5 μl/min. Unreacted groups were blocked by the injection of 35 μl of ethanolamine-HCl, pH 8.5, at 5 μl/min and the mAbs were injected at constant flow rate (5 μL/min).

Preparation and purification of rabbit anti-β-BuTx antibodies

Native β-BuTx (20 μg/0.3 ml of PBS, pH 7.4) was emulsified with equal volumes of complete Freund's adjuvant and injected intracutaneously into male New Zealand rabbits. Subsequent injections were made in incomplete Freund's adjuvant at one month interval. The rabbits were test bleed after seven days of the booster injection. The presence of reactive antibodies were measured by ELISA. The anti-β-BuTx antibodies were concentrated by ammonium sulphate (40%) precipitation and the IgG fraction was purified by passing through a protein A-Sepharose column. The rabbit anti-β-BuTx antibodies were conjugated with urease using 0.1% glutaraldehyde as the cross-linking agent.

Sandwich ELISA

Sandwich ELISA procedure was optimised for the detection/quantitation of β-BuTx. The wells of microtitre PVC plates were coated with mouse anti-mouse IgG (2 μg/mL) and the wells were incubated with mAb 15 (1 μg/mL). After blocking with 1% skim-milk solution in PBS, the wells were incubated with different concentrations of β-BuTx (0.078-10 ng/mL) was diluted in PBS, pH 7.4 . Rabbit anti-β-BuTx (1 μg/mL) antibodies was used as the detector antibodies. After washing the plates were incubated with HRP-conjugated goat anti-rabbit immunoglobulin 1:2000 in 0.5% BSA in PBS-Tween. OPD was used as a substrate.

Results

Production of monoclonal antibodies (mAbs) specific to β-BuTx

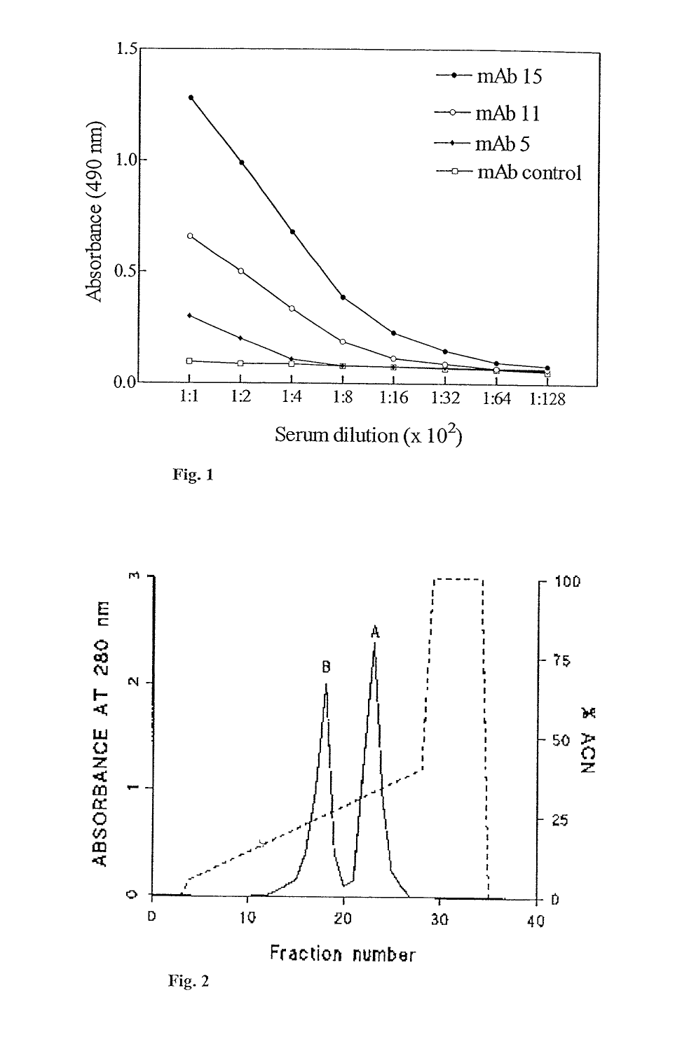

Fusion of splenocytes with P3 U1 myeloma cells led to hybrid growth in 780 out of 960 wells containing the selective hypoxanthine, aminopterin, thymidine (HAT) medium. Approximately 10 days after fusion, hybridoma supernatants were tested for secretion of anti-β-BuTx antibodies by ELISA. One hundred and forty three wells contained hybridomas which secreted antibodies specific to β-BuTx. These were subcultured and 3 hybridomas presenting the highest secreting activity were established, and the mAbs secreted by these hybridomas were designated number 5, 11 and 15, respectively. Isotyping revealed that mAbs 11 and 15 belonged to IgG1 subclass and mAb 5 to IgM class. In all three mAbs the light chains were composed of κ chains. The reactivity of mAbs 5, 11 and 15 with that of β-BuTx is shown in FIG. 1.

Epitope analysis by ELISA

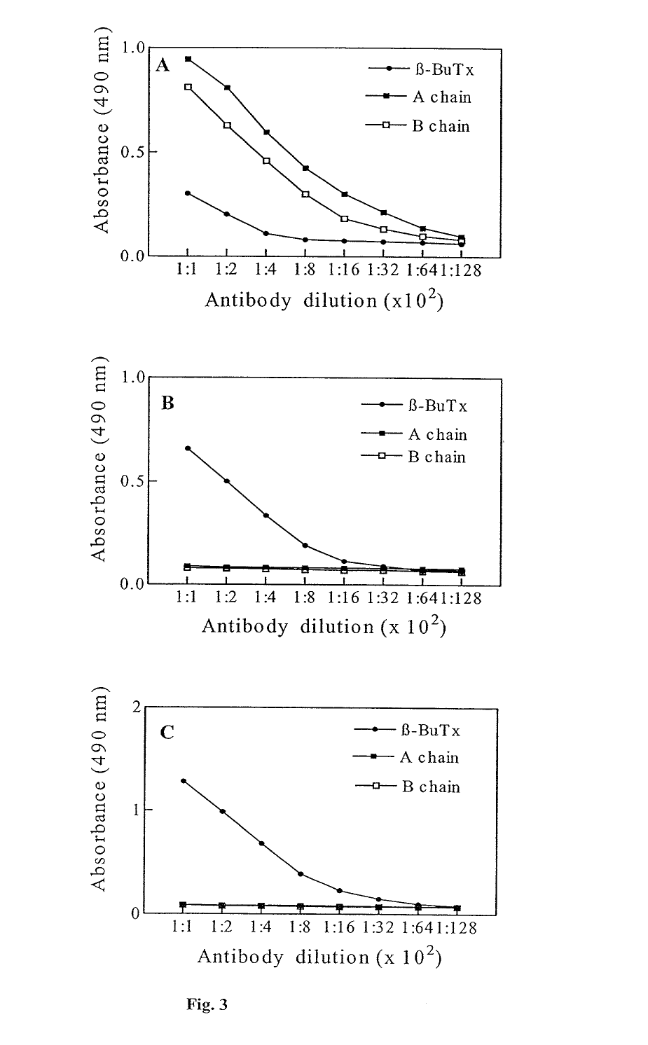

In order to ascertain the location of epitopes recognized by the three mAbs, the interchain disulfide bond between A and B chains of β-BuTx was reduced with β-mercaptoethanol. The cleavage product was separated by reverse phase HPLC (FIG. 2) and the two chains were identified by automatic Edman degradation method. The reactivity of the mAbs (5, 11 and 15) with that of β-BuTx, A chain and B chain was tested by ELISA (FIG. 3). MAbs 11 and 15 recognized only the intact native β-BuTx, they did not react with reduced subunits (FIGS. 3b and c). On the contrary, mAb 5 showed strong reactivity to reduced subunits and less reactivity to native β-BuTx (FIG. 3a). The absence of reactivity between the two mAbs and the reduced subunits suggested that mAbs 11 and 15 were raised against conformational epitopes.

Epitope analysis by BIAcore system

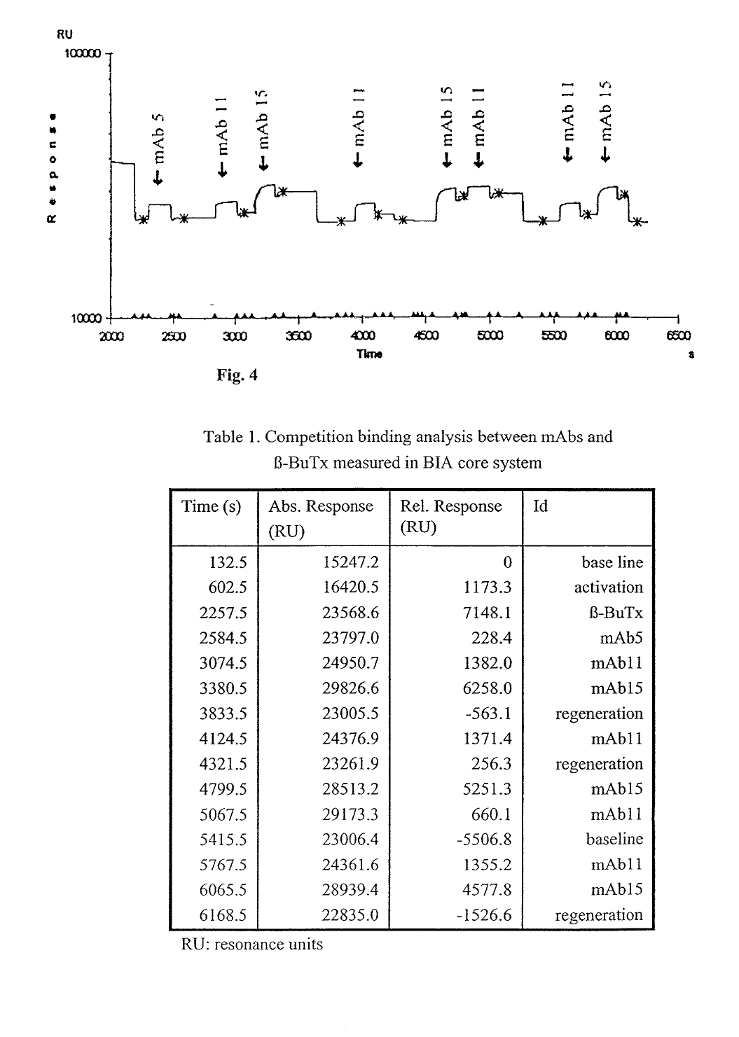

In order determine whether the three mAbs react with the same or different antigenic determinants in β-BuTx, they were examined by antibody competition test in BIA core studies. The three mAbs showed different binding characteristics (FIG. 4 and Table 1). From the results it is apparent that mAb 5 recognise a unique site of the toxin. On the contrary mAbs 11 and 15 bind on the overlapping region of an antigenic site.

Among the three antibodies, mAb 15 showed strong reactivity (FIGS. 1, 4 and Table 1) to the native toxin whereas mAbs 11 and 5 showed moderate and least reactivity, respectively. MAb 15 which showed high affinity to native β-BuTx was selected as capturing mAb for the development of the immunosensors of the invention.

Purity of mAb 15

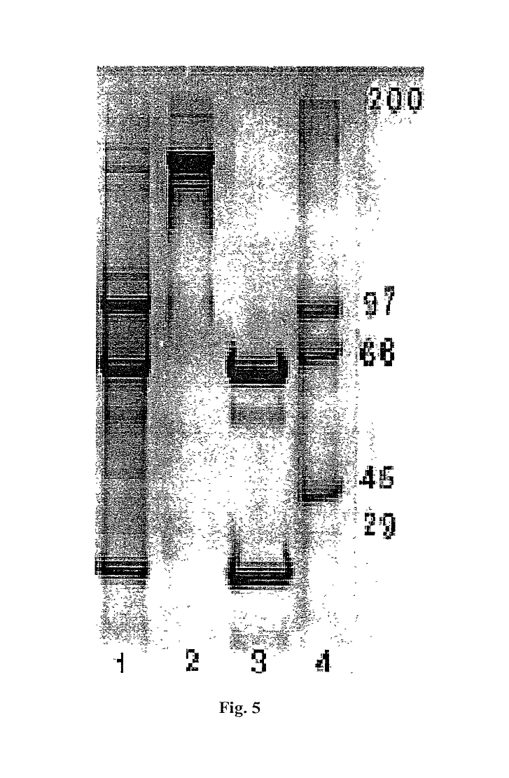

The purity of affinity purified mAb 15 was checked by electrophoresis. SDS-PAGE profile of ammonium sulphate precipitated and affinity purified IgG fractions of mAb 15 on 10% gel revealed effective purification by the protein G column (FIG. 5).

Specificity of mAb 15

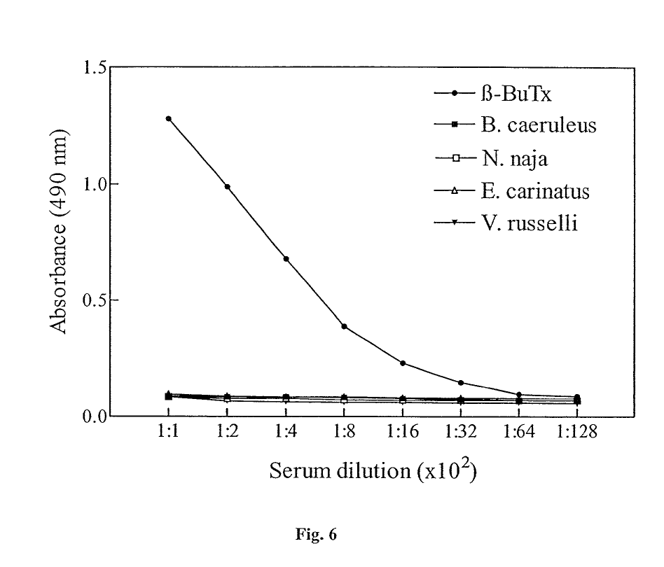

The specificity of mAb 15 antibodies was studied by ELISA (FIG. 6 ); mAb 15 showed high specificity to β-BuTx and it did not react any of the venom proteins tested.

Rabbit anti-β-BuTx antibodies

Polyclonal anti-β-BuTx antibodies were raised in rabbits and the reactivity was tested by ELISA.

Sandwich ELISA for the quantitation of β-BuTx

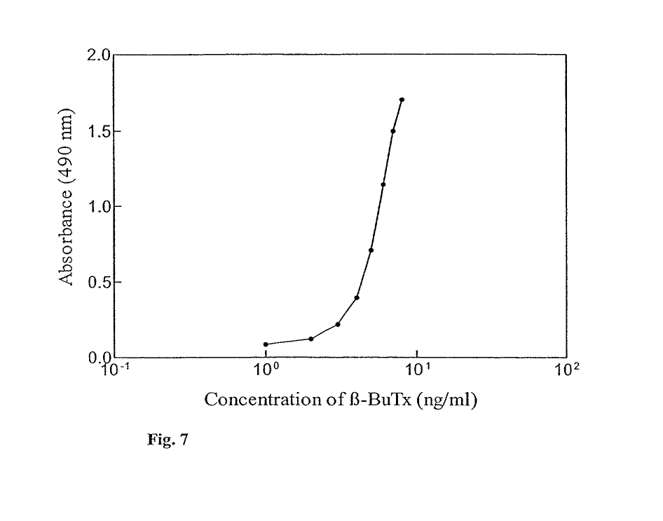

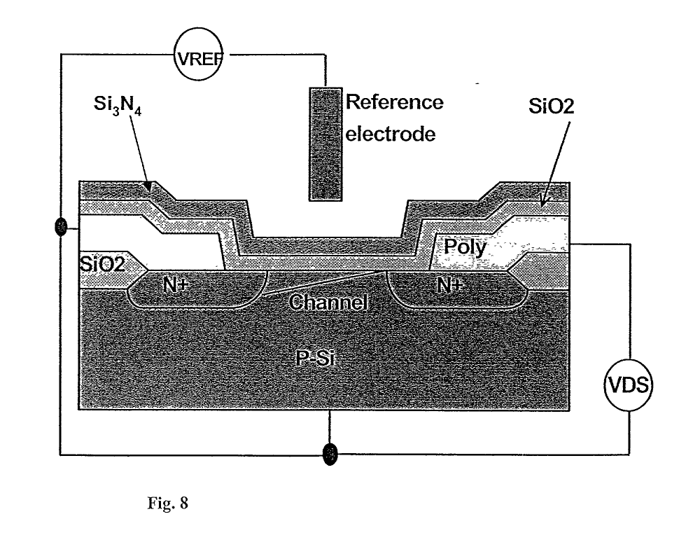

A sandwich ELISA was optimised to detect/quantitate β-BuTx. The assay can detect toxin levels as low as 0. 313 ng/ml of PBS, pH 7.4 (FIG. 7).

### EXAMPLE 2

Quantification of β-BuTx by ISFET Device

Methods

ISFET chips having an aluminium oxide surface (i.e., the pH sensitive surface) were kindly provided by Prof. Dr. Nico. F. de Rooij (Institute of Microtechnology, University of Neuchatel, Switzerland). The gate regions of two ISFETs were silanized by dip coating the sensor surface in 0.5% silane for 30 sec and blowing it with N₂ gas preferably at an angle of about 45 degrees, followed by thermal curing at 75° C. for 16 hr. The aliphatic amino group present on the polysiloxane layer were activated with 1.0% glutaraldehyde and mAb 15 was covalently immobilised to the polysiloxane layer by dip coating the activated polysiloxane layer in PBS, pH 7.4 containing the antibody at a concentration of 2 μg/mL. The incubation time for the dip coating can be between 12 to 16 hr at 4° C., and 1 to 2 hr at 37° C.

Each layer was measured by ellipsometer. Sandwich assay procedure was used for the detection/quantitation of β-BuTx. The first ISFET was incubated with β-BuTx and the second with BSA as a negative control. The antigen antibody reaction was monitored by the addition of rabbit anti-β-BuTx antibodies conjugated to urease and urea was used as the substrate. By measuring the difference between the control and experiment, only pH changes due to urea hydrolysis was detected.

The assay principle

The sandwich assay procedure was used for the detection/quantitation of β-BuTx. Mab 15 immobilised on the ISFET gate region binds analyte (β-BuTx), which then binds urease labelled rabbit anti-β-BuTx antibody. Urea was used as a substrate. The immunosensor uses a reaction wherein urea is hydrolyzed by the urease labeled second antibody. The reaction is:

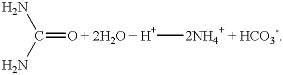

According to the reaction, the pH value in the membrane becomes high. On the other hand, on the ISFET surface with inactive antibody membrane, the above reaction does not occur and pH remains constant. Hence by measuring the differential output between two ISFETs, only pH changes due to urea hydrolysis would be detected.

Results

Antibody membrane

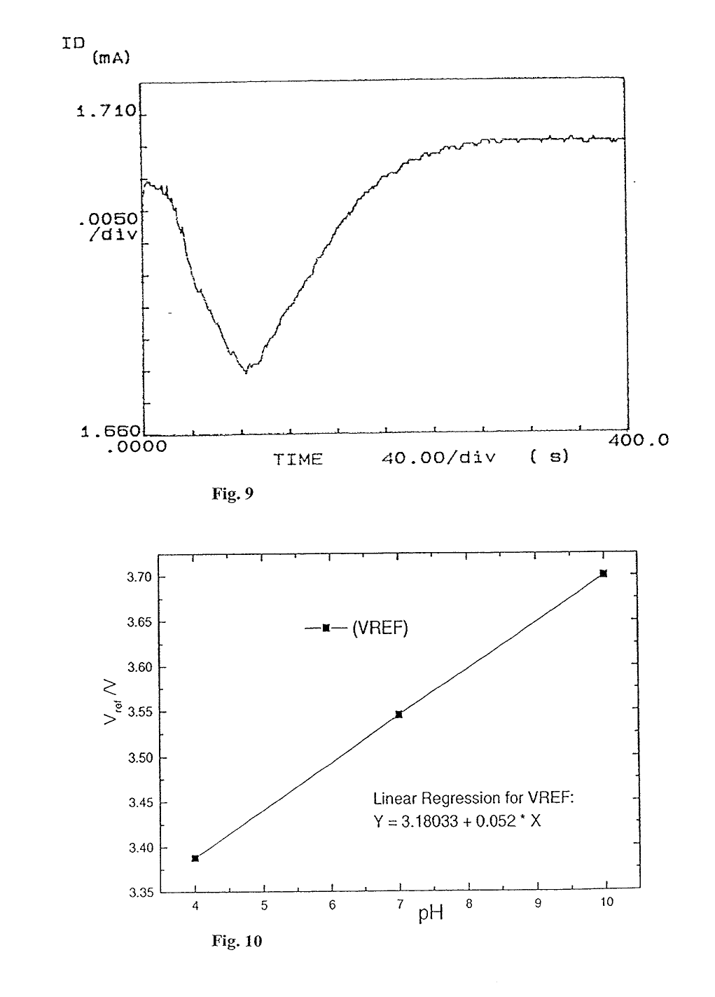

Two ISFETs were die attached on two separate printed circuit boards and were wire bonded. Except gate region, the device was encapsulated with epoxy resin (FIG. 9 is a cross section of an ISFET). The exposed gate regions were silanized and mAb 15 was covalently immobilized using glutaraldehyde. The thin film form the basis of high signal transferring capacity of the membrane to underlying transducer thereby detecting very low concentration of analyte.

Quantitation of β-BuTx by ISFET immunosensor

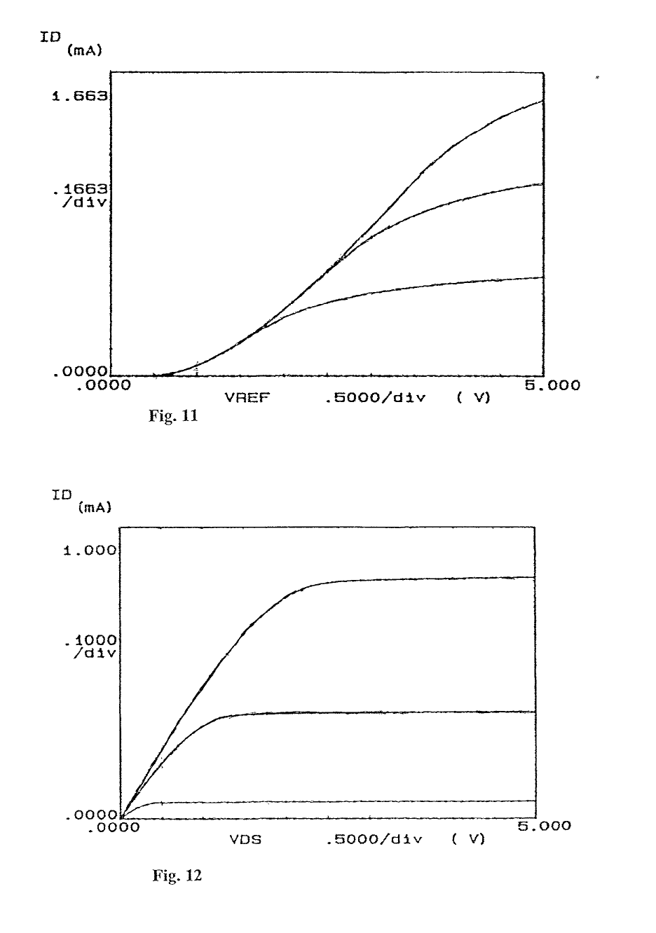

Sandwich assay method was used to detect/quantitate β-BuTx. The antigen antibody reaction was measured by the addition of urease conjugated rabbit anti-β-BuTx antibodies. When the ISFET was immersed into the substrate, the urease-urea reaction increased the pH of the membrane, which was detected by ISFET together with time response. From the preliminary experiments, we achieved the response of about −61 mV for 2.0 μg/mL and −51 mV for 0.5 μg/mL (FIG. 10). The ISFET response to the pH change was measured to be −52 mV/pH (FIG. 11). Note that the ISFET response shown in the FIG. 10 in fact shows the change of reference voltage to correct for ISFET threshold voltage change in order to keep ISFET drain current constant. The reference voltage slope of 52 mV/pH corresponds to −52 mV/pH of ISFET threshold voltage slope. Typical I_{D} vs. V_{REF} (with V_{DS} as a parameter) and I_{D} vs. V_{DS} (V_{REF} as a parameter) characteristics of an ISFET are shown in FIGS. 11 and 12, respectively.

1. A biosensor comprising an immobilised membrane adhering to a pH sensitive surface of an ion-sensitive field effect transistor by a polysiloxane matrix and comprising an analyte detection agent for detecting and/or quantifying a target analyte, the polysiloxane matrix being chosen from functional organosilanes of general formula: 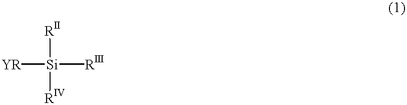
where R^{II}, R^{III}, and R^{IV}, which can be equal or different, are C₁-C₁₀ alkyl or alkoxy groups,
R=(CH₂)ₘX(CH₂)ₙ
where X is CH₂ or a mono or polycondensed aromatic group or NH or O, m and n, which can be equal or different, are whole numbers between 0 and 10, but not 0 when X is NH or O,
Y can be —NH₂ or —OH or —SH, or from functional organosilanes of general formula: 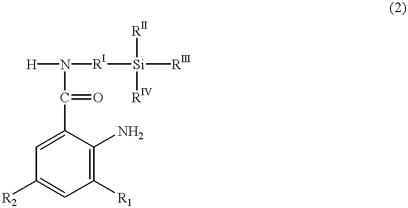
in which R₁ and R₂, which can be equal or different, are Cl, Br, CH₃, NO₂, NH₂ or H,
R^{II}, R^{III}, and R^{IV}, which can be equal or different, are C₁-C₁₀ alkyl or alkoxy groups,
R¹ can be a C₁-C₁₀ alkyl, aminoalkyl, aminoalkylaryl or alkylaryl group,
characterised in that the thickness of said membrane is less than about 100 nm.

2. The biosensor of claim 1, wherein the immobilised membrane has a thickness of between about 10 nm and about 100 nm.

3. The biosensor of claim 1, wherein the immobilised membrane has a thickness of between about 30 nm to about 90 nm.

4. The biosensor of claim 1, wherein the immobilised membrane has a thickness of between about 50 nm to about 80 nm.

5. The biosensor of claim 1, wherein the polysiloxane matrix has a thickness of between about 10 nm and about 80 nm.

6. The biosensor of claim 1, wherein the polysiloxane matrix has a thickness of between about 20 nm to about 70 nm.

7. The biosensor of claim 1, wherein the polysiloxane matrix has a thickness of between about 30 nm to about 60 nm.

8. The biosensor of claim 1, wherein the pH sensitive surface is formed of a member selected from the group consisting of aluminium oxide, silicon oxide, silicon nitride or tantalum pentoxide.

9. The biosensor of claim 1, wherein the analyte detection agent is selected from the group consisting of an antigen and an antigen-binding molecule.

10. The biosensor of claim 1, wherein the analyte detection agent is an antigen-binding molecule.

11. The biosensor of claim 1, wherein the target analyte is an antigen selected from the group consisting of a venom and a toxin.

12. The biosensor of claim 11, wherein the toxin is a bungarotoxin.

13. The biosensor of claim 12, wherein the toxin is a β-bungarotoxin.

14. A process of forming a biosensor comprising an immobilised membrane adhering to a pH sensitive surface of an ion-sensitive field effect transistor by a polysiloxane matrix and comprising an analyte detection agent for detecting and/or quantifying a target analyte, the polysiloxane matrix being chosen from functional organosilanes of general formula: 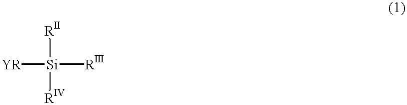
where R^{II}, R^{III}, and R^{IV}, which can be equal or different, are C₁-C₁₀ alkyl or alkoxy groups,
R═(CH₂)ₘX(CH₂)ₙ
where X is CH₂ or a mono or polycondensed aromatic group or NH or O, m and n, which can be equal or different, are whole numbers between 0 and 10, but not 0 when X is NH or O,
Y can be —NH₂ or —OH or —SH, or from functional organosilanes of general formula: 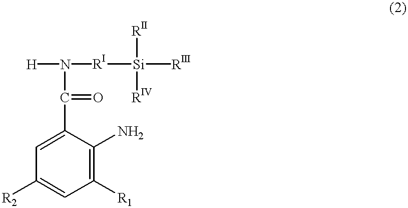
in which R₁ and R₂, which can be equal or different, are Cl, Br, CH₃, NO₂, NH₂ or H,
R^{II}, R^{III}, and R^{IV}, which can be equal or different, are C₁-C₁₀ alkyl or alkoxy groups,
R^{I} can be a C₁-C₁₀ alkyl, aminoalkyl, aminoalkylaryl or alkylaryl group,
wherein the thickness of said membrane is less than about 100 nm,
said process comprising:
applying a siloxane prepolymer to the pH sensitive surface of an ion-sensitive field effect transistor;
blowing excess siloxane prepolymer from said surface;
curing the siloxane prepolymer such that polymerisation of the silane alkoxy groups of the prepolymer takes place by hydrolysis to obtain a polysiloxane matrix;
adhering the matrix to said pH sensitive surface by reaction of other alkoxy groups with hydroxyl groups present on said surface; and
reacting an analyte detection agent with the aliphatic amino groups present on the polysiloxane matrix.

15. A process of forming a biosensor comprising an immobilised membrane adhering to a pH sensitive surface of an ion-sensitive field effect transistor by a polysiloxane matrix and comprising an analyte detection agent for detecting and/or quantifying a target analyte, the polysiloxane matrix being chosen from functional organosilanes of general formula: 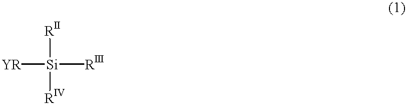
where R^{II}, R^{III}, and R^{IV}, which can be equal or different, are C₁-C₁₀ alkyl or alkoxy groups,
R═(CH₂)ₘX(CH₂)ₙ
where X is CH₂ or a mono or polycondensed aromatic group or NH or O, m and n, which can be equal or different, are whole numbers between 0 and 10, but not 0 when X is NH or O,
Y can be —NH₂ or —OH or —SH, or from functional organosilanes of general formula: 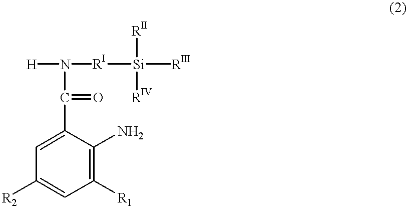
in which R₁ and R₂, which can be equal or different, are Cl, Br, CH₃, NO₂, NH₂ or H,
R^{II}, R^{III}, and R^{IV}, which can be equal or different, are C₁-C₁₀ alkyl or alkoxy groups,
R^{I} can be a C₁-C₁₀ alkyl, aminoalkyl, aminoalkylaryl or alkylaryl group,
wherein the thickness of said membrane is less than about 100 nm,
said process comprising:
applying a siloxane prepolymer to the pH sensitive surface of an ion-sensitive field effect transistor;
blowing excess siloxane prepolymer from said surface;
curing the siloxane prepolymer such that polymerisation of the silane alkoxy groups of the prepolymer takes place by hydrolysis to obtain a polysiloxane matrix;
adhering the matrix to the pH sensitive surface by reaction of other alkoxy groups with hydroxyl groups present on said surface;
activating the aliphatic amino groups present on the polysiloxane matrix by bifunctional coupling agents; and
reacting an analyte detection agent with the activated amino groups of the polysiloxane matrix.

16. The method of claim 14 or claim 15, wherein a jet of compressed gas is used to blow the excess siloxane prepolymer from the pH sensitive surface.

17. The method of claim 16, wherein the gas is selected from the group consisting of nitrogen, a noble gas, and air.

18. The method of claim 16, wherein the jet of compressed gas is blown at an angle of between 10 degrees and 70 degrees to the said surface.

19. An antigen-binding molecule that is immuno-interactive with a β-bungarotoxin.

20. A method of detecting the presence or absence of a bungarotoxin in a patient, comprising:
isolating a biological sample from the patient, contacting the biological sample with an antigen-binding molecule that is immuno-interactive with said bungarotoxin, and detecting the presence of a complex comprising the said antigen-binding molecule and the bungarotoxin.

21. The method of claim 20, wherein the antigen-binding molecule is an anti-bungarotoxin monoclonal antibody.

22. A kit comprising the biosensor of claim 1, together with a second analyte detection agent having an enzyme associated therewith, wherein the enzyme catalyses a reaction in which ions are formed from neutral molecules.

23. The kit of claim 22, wherein the enzyme is selected from the group consisting of urease, penicillinase, esterases, hydrolases, amino acid oxidase and glucose oxidase.

24. A kit detecting and/or quantifying a bungarotoxin, comprising the antigen binding molecule of claim 19, together with one or more reagents selected from the group consisting of reagents for detection of reporter molecules, positive and negative controls, washing solutions, and dilution buffers.

25. A composition for treatment or prophylaxis snake envenomation caused by a Bungarus species, comprising the antigen-binding molecule of claim 19, together with a pharmaceutically acceptable carrier.

26. The composition of claim 25, wherein the Bungarus species is Bungarus multicinctus.
